(19) United States
(12) Patent Application Publication
(10) Pub. No.: US 2020/0030263 A1
(43) Pub. Date: Jan. 30, 2020

(54) METHODS AND COMPOSITIONS FOR PROMOTING WOUND HEALING WITH DECREASED SCAR FORMATION AFTER GLAUCOMA FILTRATION SURGERY

(71) Applicant: United States Government as Represented by the Department of Veterans Affairs, Washington, DC (US)

(72) Inventor: Rajiv R. Mohan, Columbia, MO (US)

(21) Appl. No.: 16/596,510

(22) Filed: Oct. 8, 2019

Related U.S. Application Data
(63) Continuation of application No. 16/016,458, filed on Jun. 22, 2018.
(60) Provisional application No. 62/523,673, filed on Jun. 22, 2017.

(51) Int. Cl. A61K 31/165; A61K 31/506; A61K 31/4045; A61P 27/02; A61K 31/437; A61K 9/00; A61P 17/02; A61K 31/4406

(52) U.S. Cl. CPC A61K 31/165; A61K 31/506; A61K 31/4045; A61K 31/4406; A61K 31/437; A61K 9/0048; A61P 17/02; A61P 27/02

(57) ABSTRACT

Disclosed is a method of promoting wound healing with reduced scarring after glaucoma filtration surgery in a mammalian subject in need thereof, which involve the use of a HDAC inhibitor (HDACi), such as, but not limited to, suberoylanilide hydroxamic acid (SAHA).

### CROSS REFERENCE TO RELATED APPLICATIONS

This application is a continuation of U.S. application Ser. No. 16/016,458, filed Jun. 22, 2018, which claims the benefit of U.S. Provisional Patent Application No. 62/523,673, filed Jun. 22, 2017, which is incorporated herein by reference in its entirety.

This invention was made with Government Support from the Department of Veterans Affairs and the National Eye Institute (Grant No. 2RO1EY017294-06). The Government has certain rights in the invention.

Throughout the present specification certain references are referred to by superscript citation numbers, which numbers refer to the references enumerated in the list included herein before the claims. These enumerated references, and others which may be cited herein, are intended to describe the state of the art and are incorporated herein by reference in their entireties.

### BACKGROUND OF THE INVENTION

### Field of the Invention

The present invention is related to the treatment of ophthalmic diseases in mammalian subjects, including humans.

### Related Art

Glaucoma is the second leading cause of blindness according to the world health organization. In the United States alone; glaucoma affects approximately 2.2 million people. The major goal of glaucoma therapy is to reduce intraocular pressure (TOP) to levels considered safe for the optic nerve to preserve visual function. Currently, several topical drugs capable of reducing intraocular pressure are used for glaucoma treatment. However, a large number of patients do not respond adequately to topical drug therapy to reduce IOP.

The most preferred treatment for such patients is glaucoma filtration surgery (GFS). A major deterrent to the success of GFS is caused by aberrant post-operative wound healing resulting in excessive ECM synthesis leading to fibrosis over filtering bleb. Development of fibrosis and collagen deposit at sclerotomy site compromises bleb's proper functioning and disable its ability to maintain non-pathologic reduced IOP.

To prevent this common complication, cytotoxic drugs such as mitomycin C (MMC) and 5-fluorouracil are frequently utilized intra-operatively and in clinical practice to reduce scar formation caused by GFS. Though these drugs are effective in preventing ocular fibrosis and improving the outcome of filtration surgery, they are known to cause sight-threatening complications including wide spread cell death, bleb leak, hypotony, and/or endophthalmitis.

Glaucoma filtration surgery initiates a cascade of events including blood exudation, fibrin deposit, recruitment of inflammatory cells and release of cytokines/growth factors. The released cytokines alter the gene expression of proliferative, cytoskeletal and matrix proteins, thus leading to fibrosis. Epigenetic regulations play a critical role, which comprises regulation of gene expression by methylation/acetylation of DNA and histone proteins. Histone acetylation regulates gene expression by altering DNA structure thus influencing DNA binding to various transcription factors.

Histone deacetylase inhibitors (HDACi) represent a new class of pharmacological agents that can modulate gene expression by increasing histone acetylation of chromatin and other non-histone proteins. HDAC inhibitors have been shown to have pleiotropic anti-fibrotic effect in vivo in a wide variety of animal models of skin, liver, lung and heart fibrosis. Initially developed as anticancer drugs, they are increasingly being shown to be effective in treating fibrosis.

Although several HDACi molecules are at various stages of preclinical and clinical development, suberoylanilide hydroxamic acid (SAHA; also known as MK-0683, VOR, and volinostat) is the only HDACi currently approved by the FDA for use in human patients, and is available commercially as Zolinza® (Merck & Co., Inc.).

In other reports, the anti-fibrotic effect of SAHA on laser-surgery induced corneal scarring in vivo, after a period of one month (Sharma A, et al., Trichostatin A inhibits corneal haze in vitro and in vivo. Invest Ophthalmol Vis Sci. 2009; 50:2695-701; Tandon A, et al. SAHA: a potent agent to prevent and treat laser-induced corneal haze. J Refract Surg. 2012; 28:285-90). Unlike other anti-fibrotic agents, SAHA is relatively non-toxic and does not affect the viability or proliferation of corneal fibroblasts (see Sharma et al. 2009; Tandon et al. 2012; Donnelly et al., Suberoylanilide hydroxamic acid (SAHA): its role on equine corneal fibrosis and matrix metalloproteinase activity. Vet Ophthalmol. 2014; 17 Suppl 1:61-8; Bosiack et al., Efficacy and safety of suberoylanilide hydroxamic acid (SAHA) in the treatment of canine corneal fibrosis. Vet Ophthalmol. 2012; 15:307-14).

However, methods and pharmaceutical compositions to promote ocular wound healing are needed after GFS, while effectively preventing bleb fibrosis, and without significant adverse side effects. This the present invention provides.

### SUMMARY OF THE INVENTION

The present invention is directed to a method of promoting wound healing with reduced scarring after glaucoma filtration surgery in a mammalian subject in need thereof. The method involves administering an effective amount of a HDAC inhibitor (HDACi) to said subject.

We have demonstrated in the examples hereinafter the efficacy of the present invention by showing, inter alia, that suberoylanilide hydroxamic acid (SAHA), an HDAC inhibitor (HDACi) prevents excessive wound healing and scar formation in a rabbit model of glaucoma filtration surgery (GFS).

Another aspect of the invention is a pharmaceutical composition, comprising a HDACi, and a pharmaceutically acceptable carrier or excipient suitable for ophthalmic use.

Accordingly, provided herein is a method of promoting wound healing with reduced scarring after glaucoma filtration surgery in a mammalian subject in need thereof, comprising administering an effective amount of a HDAC inhibitor (HDACi) to said subject. In one embodiment, the HDACi is suberoylanilide hydroxamic acid (SAHA), or a derivative thereof. In this embodiment, SAHA inhibits fibroblast migration and activation. In another aspect of this embodiment, myofibroblast formation is inhibited. In a particular embodiment, myofibroblast formation is inhibited while preserving cell viability.

In other embodiments, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

Also provided herein is a pharmaceutical composition, comprising a HDACi, and a pharmaceutically acceptable carrier or excipient suitable for ophthalmic use. In one embodiment, the HDACi is suberoylanilide hydroxamic acid (SAHA) or a derivative thereof, or a physiologically acceptable salt thereof.

In another embodiment, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

Also provided herein is a method of preventing or reducing corneal haze formation long-term, after photorefractive keratectomy (PRK) surgery in a mammalian subject in need thereof, comprising administering an effective amount of a HDAC inhibitor (HDACi) to said subject, wherein said corneal haze formation is prevented or reduced long-term. In one embodiment, the HDACi is suberoylanilide hydroxamic acid (SAHA), or a derivative thereof. In particular embodiments, the corneal haze formation is prevented or reduced for a period selected from the group consisting of: greater than 1 month; greater than or equal to 2 months, greater than or equal to 3 months, greater than or equal to 4 months, greater than or equal to 5 months, greater than or equal to 6 months, greater than or equal to 7 months, greater than or equal to 8 months, greater than or equal to 9 months, greater than or equal to 10 months, greater than or equal to 11 months, greater than or equal to 12 months. In a particular embodiment, the corneal haze formation is prevented or reduced for a period greater than or equal to 4 months, and the endothelial cell phenotype and density is not compromised. In another embodiment, the corneal haze formation is prevented or reduced for a period greater than or equal to 4 months, and the density of keratocytes is not reduced. In other embodiments, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1A-1I shows suberoylanilide hydroxamic acid or mitomycin C treatment improves bleb morphology after Glaucoma Filtration Surgery (GFS). Representative stereomicroscopic images were captured at day 3, 7 and 14 after glaucoma filtration surgery showing bleb characteristics in no-treatment control (FIG. 1A-1C), suberoylanilide hydroxamic acid (SAHA)-treated (FIG. 1D-1F) and mitomycin C (MMC)-treated (FIG. 1G-1I) rabbit eyes. Suberoylanilide hydroxamic acid or mitomycin C-treated rabbit eyes showed bigger bleb area (outlined in black dotted lines). Scale bar=3 mm.

FIG. 2A-2B shows quantitation of bleb area and length. Quantification of bleb length (FIG. 2A) and bleb area (FIG. 2B) at day 3, 7 and 14 after glaucoma filtration surgery in no treatment control, suberoylanilide hydroxamic acid- and mitomycin C-treated rabbit eyes. Suberoylanilide hydroxamic acid- or mitomycin C-treated rabbits showed significantly bigger blebs. σ p<0.05, τ p<0.01, Ψ p<0.001 compared to control.

FIG. 3 shows that suberoylanilide hydroxamic acid decreases bleb vascularity. Quantification of bleb vascularity at day 3, 7 and 14 after glaucoma filtration surgery in no treatment control, suberoylanilide hydroxamic acid and Mitomycin C treated rabbit eyes. Both suberoylanilide hydroxamic acid and Mitomycin C significantly reduced the bleb vascularity. τ p<0.01 compared to control, Ψ p<0.001 compared to control and suberoylanilide hydroxamic acid.

FIG. 4 shows that suberoylanilide hydroxamic acid or mitomycin C reduces intraocular pressure (TOP). The TOP was measured at day 3, 7 and 14 after glaucoma filtration surgery in no treatment control, suberoylanilide hydroxamic acid- or mitomycin C-treated rabbits. Suberoylanilide hydroxamic acid- or mitomycin C-treated rabbits had lower TOP at day 7 and day 14 as compared to no treatment control but the decrease is not statistically significant.

FIG. 5A-5D shows that suberoylanilide hydroxamic acid decreases collagen deposition at the site of GFS. Representative images showing H&E (FIG. 5A, FIG. 5B) and Masson's trichrome (FIG. 5C, FIG. 5D) staining in no treatment control (FIG. 5A, FIG. 5C) and suberoylanilide hydroxamic acid treated (FIG. 5B, FIG. 5D) rabbit eyes. The tissues were collected at day 14 after the glaucoma filtration surgery. The H&E staining of suberoylanilide hydroxamic acid treated tissues sections (FIG. 5B) shows loosely arranged less fibrous conjunctival tissues whereas no treatment control tissues (FIG. 5A) are densely packed with fibrous deposit. Masson's trichrome staining shows prominent collagen deposit (blue color) in control tissues (FIG. 5C) whereas collagen deposit is notably decreased in suberoylanilide hydroxamic acid treated rabbit tissues (FIG. 5D). Scale bar=400 μm.

FIG. 6A-6F demonstrate that suberoylanilide hydroxamic acid treatment increases acetylation of histones in human corneal fibroblasts and Rabbit conjunctiva tissues. FIG. 6A, FIG. 6C, and FIG. 6D show results for human corneal fibroblasts cell treated with suberoylanilide hydroxamic acid (2.5 μM) for the indicated times, and FIG. 6B, FIG. 6 E, and FIG. 6F show results for rabbit conjunctiva injected with suberoylanilide hydroxamic acid (50 μM) for the indicated times, as analyzed by Western blot using anti-Ac-Histone H3, anti-Ac-Histone H4 and anti-β-actin antibodies.

FIG. 7 shows that suberoylanilide hydroxamic acid treatment decreases f-actin assembly. Representative images were taken of rabbit ocular tissue sections were immunofluorescence-stained for f-actin (marker for activated fibroblasts and myofibroblast) in no treatment control, suberoylanilide hydroxamic acid-treated and mitomycin C-treated tissue sections (data not shown). The tissues were collected at day 14 after the glaucoma filtration surgery. The Graph depicting the images (FIG. 7) shows a significant (* p<0.01) decrease in the f-actin stained area in the suberoylanilide hydroxamic acid- and mitomycin C-treated rabbit tissues. Discontinuous and highly sparse DAPI nuclear staining of the conjunctival epithelium in the mitomycin C-treated rabbit tissue sections was observed.

FIG. 8 shows that suberoylanilide hydroxamic acid reduces myofibroblast conversion at the site of GFS. Representative images were taken of rabbit ocular tissue sections. are presented of the site of sclerotomy showing immunofluorescence staining for a smooth muscle actin (αSMA, a myofibroblast marker) in no treatment control, suberoylanilide hydroxamic acid-treated and mitomycin C-treated (data not shown). The Graph depicting the images (FIG. 8) shows a significant (* p<0.01) decrease in the αSMA stained area in the suberoylanilide hydroxamic acid- and mitomycin C-treated rabbit tissues. Discontinuous and highly sparse DAPI nuclear staining of the conjunctival epithelium in the MMC-treated rabbit tissue sections was observed.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a molecule” includes a plurality of molecules; reference to “a cell” includes populations of a plurality of cells.

Reduced Scarring after Glaucoma Filtration Surgery

Provided herein is a method of promoting wound healing with reduced scarring after glaucoma filtration surgery (GFS) in a mammalian subject in need thereof, comprising administering an effective amount of a HDAC inhibitor (HDACi) to said subject. In one embodiment, the HDACi is suberoylanilide hydroxamic acid (SAHA), or a derivative thereof. In this embodiment, SAHA inhibits fibroblast migration and activation. In another aspect of this embodiment, myofibroblast formation is inhibited. In a particular embodiment, myofibroblast formation is inhibited while preserving cell viability.

In other embodiments, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

GFS remains the mainstay procedure for the clinical management of drug-refractory glaucoma. Post-operative wound healing of the scleral flap and scarring of the overlying conjunctiva are the major impediments to surgical success of GFS. We demonstrate herein that a HDAC inhibitor, e.g., SAHA, reduces post-operative scarring in the rabbit model of GFS. Bleb areas were consistently larger in the SAHA-treated groups and it was corroborated by histological findings showing decreased extracellular matrix deposit and collagen deposition. Several lines of evidence imply that histone acetylation plays a role in transcriptional regulation probably by altering chromatin structures. Chromatin fractions enriched in actively transcribed genes are also enriched in the more highly acetylated isoforms of the core histones. SAHA binds directly to the catalytic site of HDAC, inhibiting its deacetylase enzymatic activity. We demonstrate herein the beneficial effect of a HDACi on the outcome of GFS. Although the exact mechanism of their anti-fibrotic effect is not fully understood—and the present invention does not rely on any particular mechanism of action, several hypothesis have been proposed including the suppression of profibrotic genes such as CTGF or upregulation of anti-fibrotic genes such as TGIFs and SMAD7. Additionally, HDACi molecules have been shown to inhibit pro-inflammatory cytokine production and have an anti-inflammatory effect in the disease models of inflammatory bowel diseases, multiple sclerosis, and systemic lupus erythematosus. All of these biological effects of HDAC inhibition may have collectively contributed to the observed anti-fibrotic effect of SAHA in the experiments described herein. At present, four different HDACi molecules are being tested in clinical trials, and any of these can be useful in practicing the present invention. SAHA is FDA approved HDACi in clinical use.

Wound healing is a well-orchestrated event in vivo, which involves local and recruited progenitor and differentiated cells, growth factors and cytokines, extracellular matrix (ECM) and relevant enzymes that modify molecular components of the matrix. Due to the high metabolic activity at a wound site, there is an increasing demand for oxygen and nutrients. Angiogenesis is crucial for wound healing, which supports new tissue growth by allowing adequate distribution of oxygen and nutrients for new tissues to sustain its metabolic needs. It progressively proceeds by sprouting and elongation of new capillaries from the blood vessels of the intact tissues around the wound. Fibroblasts are attracted into the wounds and within the wound bed, to produce collagen as well as glycosaminoglycan's and proteoglycans, which are major components of the extracellular matrix (ECM). Following robust proliferation and ECM synthesis, wound healing should stop when a tissue gap is filled. In addition, regression of many of the newly formed capillaries occurs, so that vascular density of the wound returns to normal. In some cases proliferation proceeds longer than needed leading to elevated scars or even scars whose tissue tends to overgrow. The fibroblast is considered the pivotal cell in pathologic scarring because of its role in matrix deposition and remodeling and HDAC inhibitor showed suppression of neovascularization through alteration of genes directly involved in angiogenesis.

Wound healing after GFS involves activation and migration of conjunctival and tenon's capsular fibroblasts to the sclerostomy site followed by their differentiation into myofibroblasts. Myofibroblasts are highly contractile and metabolically active cells causing wound closure by direct contraction and excessive ECM deposition. In the experiments described herein, SAHA-treated eyes showed significantly less staining for activated fibroblasts and myofibroblasts in the sclera and conjunctiva surrounding the surgery site. These observations indicate that SAHA attenuates post GFS scarring through inhibition of fibroblast migration and activation as well as attenuation of myofibroblast formation. These results are supported by the previous studies from our lab showing that SAHA is capable of inhibiting differentiation of cultured rabbit, equine, canine and human corneal fibroblasts to myofibroblasts. See, e.g., Tandon et al., SAHA: a potent agent to prevent and treat laser-induced corneal haze. J Refract Surg. 2012; 28:285-90; Donnelly et al., Suberoylanilide hydroxamic acid (SAHA): its role on equine corneal fibrosis and matrix metalloproteinase activity. Vet Ophthalmol. 2014; 17 Suppl 1:61-8; Bosiack et al., Efficacy and safety of suberoylanilide hydroxamic acid (SAHA) in the treatment of canine corneal fibrosis. Vet Ophthalmol. 2012; 15:307-14; each of which are incorporated herein by reference in their entirety. Furthermore, in the earlier studies a 50 μM or higher dose of SAHA did not decrease cellular viability of cultured corneal fibroblasts. Therefore, we infer that in the present study SAHA did not cause any cytotoxicity to tenon or conjunctival fibroblasts. Lack of inflammation, corneal edema, opacity, endophthalmitis, or cataract formation in SAHA-treated rabbit eyes supports this conclusion as well. The lack of cytotoxic effect of SAHA is in stark contrast to currently used drugs MMC or 5-fluorouracil which presumably inhibit GFS by causing myofibroblast cell death. The nonselective cytotoxic effect of these drugs accounts for the potentially sight-threatening side effects whereas SAHA appears to inhibit myofibroblast formation while preserving cell viability.

Previous studies have shown that drug application before sclerostomy, minimizes drug spillage into the anterior chamber which translates to less ciliary body toxicity. Therefore, we preferred to choose preoperative subconjunctival application of SAHA instead of intraoperative topical application. A one-time intraoperative application of either MMC or 5-fluorouracil during GFS is standard practice in clinical setting. In the experiments described herein, our decision to administer one SAHA application was tailored to reflect this current clinical practice. SAHA has a short plasma half-life but based on the data at hand, one time SAHA application seems potent enough to improve GFS outcome. There is no simple and apparent explanation for these observations. Surgical trauma unfolds a cascade of interrelated events leading to a vicious cycle of excessive wound healing. It is possible that a single SAHA application potently inhibits these early events and can block the entire fibrotic cascade. Alternatively, SAHA may have some local tissue binding to show an extended release profile. It is also possible that the gene transcription changes induced by a single SAHA dose may last for a few days which can counter the ongoing fibrotic process.

Preventing or Reducing Corneal Haze Formation Long-Term, after Photorefractive Keratectomy (PRK) Surgery

Also provided herein is a method of preventing or reducing corneal haze formation long-term, after photorefractive keratectomy (PRK) surgery in a mammalian subject in need thereof, comprising administering an effective amount of a HDAC inhibitor (HDACi) to said subject, wherein said corneal haze formation is prevented or reduced long-term. In one embodiment, the HDACi is suberoylanilide hydroxamic acid (SAHA), or a derivative thereof.

As used herein the term “long-term” or “stable” as used in the context of the invention method of reducing corneal haze formation after PRK, refers to any time period that is greater than 4 weeks to about 1 month. In particular embodiments, the corneal haze formation is prevented or reduced for a period selected from the group consisting of: greater than 1 month; greater than or equal to 2 months, greater than or equal to 3 months, greater than or equal to 4 months, greater than or equal to 5 months, greater than or equal to 6 months, greater than or equal to 7 months, greater than or equal to 8 months, greater than or equal to 9 months, greater than or equal to 10 months, greater than or equal to 11 months, greater than or equal to 12 months.

The long-term or stable effectiveness of the invention methods can be assessed as described herein, for example, by the endothelial cell phenotype and density not being compromised. Another way to assess the long-term or stable effectiveness of the invention method is by determining that the density of keratocytes is not reduced after treatment, at time points greater than 4 weeks or 1 month. Yet another way to assess the long-term or stable effectiveness of the invention method is by determining that apoptosis is not induced in any cell type after treatment, at time points greater than 4 weeks or 1 month. Accordingly, in a particular embodiment, the corneal haze formation is prevented or reduced for a period greater than or equal to 4 months, and the endothelial cell phenotype and density is not compromised. In another embodiment, the corneal haze formation is prevented or reduced for a period greater than or equal to 4 months, and the density of keratocytes is not reduced. In other embodiments, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

Adjunct therapy is needed to reduce corneal haze after PRK, especially with higher diopter treatments. Our group has previously demonstrated that HDAC inhibitors effectively reduce corneal haze and scarring in vivo in the rabbit cornea without causing significant acute side effects. See, e.g., Sharma A, et al., Trichostatin A inhibits corneal haze in vitro and in vivo. Invest Ophthalmol Vis Sci. 2009; 50(6):2695-2701; Tandon A et al., Vorinostat: a potent agent to prevent and treat laser-induced corneal haze. J Refract Surg. 2012; 28(4):285-90; Donnelly K S et al., Suberoylanilide hydroxamic acid (vorinostat): its role on equine corneal fibrosis and matrix metalloproteinase activity. Vet Ophthalmol. 2014; 17 Suppl 1:61-68; and Gronkiewicz K M et al., Molecular mechanisms of suberoylanilide hydroxamic acid in the inhibition of TGF-β1-mediated canine corneal fibrosis. Vet Ophthalmol. 2016; 19(6):480-487; each of which are incorporated herein by reference in their entirety. In accordance with the present invention, we found adjunct topical SAHA and MMC application after PRK significantly prevented corneal haze and decreased the pro-fibrotic biomarkers in vivo in rabbits. Although haze inhibition by SAHA was less than MMC, this difference was statistically insignificant and appeared clinically irrelevant based on the slit-lamp subjective analysis. The most remarkable findings of the present study were the detection of significantly reduced cytotoxicity and enhanced safety profile by SAHA compared to MMC. SAHA application demonstrated markedly improved keratocyte viability and phenotype, reduced keratocyte and endothelial apoptosis and strikingly better endothelial cellular morphology. These results indicate that topical adjunct SAHA application after PRK would be a safer alternative to MMC in preventing post-PRK corneal haze.

The corneal wound healing response plays a central role in the outcome of refractive surgery. Pharmacologically broad acting agents, specifically steroids and MMC, are most commonly used to control post-PRK scarring. Increasingly precise targeted control of the corneal wound healing response will lead to faster recovery times, more accurate refractive outcomes and decreased complication rates. In vitro analysis reveals that expression of α-SMA in stress fibers confers to the differentiated myofibroblast at least a two-fold stronger contractile activity compared with α-SMA-negative fibroblasts. We found many α-SMA expressing myofibroblasts after PRK in the anterior stroma of rabbit cornea. It is likely that these cells contribute to corneal scar formation and refractive outcome of the procedure. The clearance of α-SMA expressing cells from the anterior stroma of MMC treated corneas suggest the decrease in myofibroblasts due to MMC toxicity. This toxicity is also responsible for a diminished keratocyte population available for conversion to myofibroblasts at the site of injury.

The literature suggests that during laser photo disruption some cells are vaporized instantly, while cells in close proximity went into a slow involution form of cell death, known as apoptosis. Myofibroblasts may undergo apoptosis or undergo transdifferentiation back to a progenitor cell. The level of keratocyte apoptosis distribution, along with activated stromal keratocytes repopulation, are likely contributors of corneal wound healing associated with variability and regression after PRK.

Previous studies demonstrated that topical application of MMC after PRK in rabbits not only decreased keratocyte density due to apoptosis at the wound site, but it significantly delayed keratocyte repopulation and activation in the anterior stroma with normal epithelial cell differentiation. In accordance with the present invention, we observed a similar cytotoxicity pattern in MMC treated rabbit corneas in which several TUNEL+ cells at shorter times and low DAPI-stained nuclei at longer times in the anterior stroma were observed. Contrary to this, SAHA treatment did not show such damage to the anterior stroma. These findings indicate that SAHA has a superior safety profile than MMC in the treatment of corneal haze after PRK.

The corneal endothelial cells do not replicate in humans and therefore their preservation is important for corneal transparency and normal functioning. In accordance with the present invention, topical application of SAHA did not cause apoptosis in endothelial cells, and in addition showed a typical polygonal morphology and cellular density similar to untreated control corneas up to 4-months, the longest tested time point. The conflicting literature on the effects of MMC on human corneal endothelial cells exist. A nonrandomized controlled trial showed that the prophylactic use of MMC (0.02%; 10-50 seconds) inhibited haze formation but caused significant loss of corneal endothelial cells. In contrast, other studies have shown that the administration of 0.02% MMC topically applied to the cornea for 12 seconds and 40 seconds following PRK did not have a significant effect on qualitative morphometric parameters or quantitative endothelial cell density.

In summary, the efficacy and short- and long-term safety of MMC and SAHA has been compared. The results provided in accordance with the present invention indicate that single topical adjunct use of SAHA after −9D PRK efficaciously prevents post-operative corneal haze without reducing keratocyte population or compromising corneal endothelial cells in vivo. In accordance with the present invention, SAHA offers an alternative to MMC for preventing corneal haze in patients undergoing PRK surgery.

The phrase “mammalian subject in need thereof” means a mammal, including a human patient, who has been determined to have a medical need for, or who has undergone, a glaucoma filtration surgery (GFS) procedure.

Administering the HDACi to the subject or patient can be before, simultaneous with, or subsequent to, the GFS procedure.

As used herein, the phrase “effective amount” means a dose or quantity of an HDAC inhibitor, that eliminates or ameliorates scarring after GFS in a subject or patient.

The HDAC inhibitor can be any molecule that inhibits the biological activity of a mammalian histone deacetylase (HDAC), including a human HDAC. Examples of useful HDAC inhibitors include, but are not limited to, suberoylanilide hydroxamic acid (SAHA), Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives or modifications or physiologically acceptable salt(s) of any HDACi molecule, that retain HDAC-inhibitory biological activity.

The term “derivative,” refers to HDAC inhibitors that are modified by covalent conjugation to other therapeutic or diagnostic agents or moieties, or to a label or marker (e.g., a radionuclide or one or more various enzymes), or are covalently conjugated to a protein, such as an immunoglobulin Fc domain or other “carrier” molecule, or to a polymer, such as polyethylene glycol (PEGylation) or biotin (biotinylation).

“Physiologically acceptable salt” of a composition of matter, for example a salt of a HDACi means any salt, or salts, that are known or later discovered to be pharmaceutically acceptable. Some non-limiting examples of pharmaceutically acceptable salts are: acetate salts; trifluoroacetate salts; hydrohalides, such as hydrochloride (e.g., monohydrochloride or dihydrochloride salts) and hydrobromide salts; sulfate salts; citrate salts; maleate salts; tartrate salts; glycolate salts; gluconate salts; succinate salts; mesylate salts; besylate salts; salts of gallic acid esters (gallic acid is also known as 3, 4, 5 trihydroxybenzoic acid) such as PentaGalloylGlucose (PGG) and epigallocatechin gallate (EGCG), salts of cholesteryl sulfate, pamoate salts, tannate salts, and oxalate salts.

### Pharmaceutical Compositions

Also provided herein is a pharmaceutical composition, comprising a HDACi, and a pharmaceutically acceptable carrier or excipient suitable for ophthalmic use. In one embodiment, the HDACi is suberoylanilide hydroxamic acid (SAHA) or a derivative thereof, or a physiologically acceptable salt thereof.

In another embodiment, the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

In General. The present invention also provides pharmaceutical compositions comprising a HDACi and a pharmaceutically acceptable carrier suitable for ophthalmic administration, e.g., suitable for subconjunctival, intravitreal, or topical administration, e.g., using eye drops and the like. Such pharmaceutical compositions can be configured for administration to a patient by a wide variety of delivery ophthalmic routes, e.g., subconjunctival injection, or other ocular delivery routes and/or forms of administration known in the art. The inventive pharmaceutical compositions may be prepared in liquid form, e.g., for administration via eye drops, or may be in dried powder form, such as lyophilized form.

In the practice of this invention the “pharmaceutically acceptable carrier” is any physiologically tolerated substance known to those of ordinary skill in the art useful in formulating pharmaceutical compositions, including, any pharmaceutically acceptable diluents, excipients, dispersants, binders, fillers, glidants, anti-frictional agents, compression aids, tablet-disintegrating agents (disintegrants), suspending agents, lubricants, flavorants, odorants, sweeteners, permeation or penetration enhancers, preservatives, surfactants, solubilizers, emulsifiers, thickeners, adjuvants, dyes, coatings, encapsulating material(s), and/or other additives singly or in combination. Such pharmaceutical compositions can include diluents of various buffer content (e.g., Tris-HCl, acetate, phosphate), pH and ionic strength; additives such as detergents and solubilizing agents (e.g., Tween® 80, Polysorbate 80), anti-oxidants (e.g., ascorbic acid, sodium metabisulfite), preservatives (e.g., Thimersol®, benzyl alcohol) and bulking substances (e.g., lactose, mannitol); incorporation of the material into particulate preparations of polymeric compounds such as polylactic acid, polyglycolic acid, etc. or into liposomes. The compositions can be prepared in liquid form, or can be in dried powder, such as lyophilized form. Implantable sustained release formulations are also useful, as are transdermal or transmucosal formulations. Additionally (or alternatively), the present invention provides compositions for use in any of the various slow or sustained release formulations or microparticle formulations known to the skilled artisan, for example, sustained release microparticle formulations, which can be administered via pulmonary, intranasal, or subcutaneous delivery routes. (See, e.g., Murthy et al, Injectable compositions for the controlled delivery of pharmacologically active compound, U.S. Pat. No. 6,887,487; Manning et al., Solubilization of pharmaceutical substances in an organic solvent and preparation of pharmaceutical powders using the same, U.S. Pat. Nos. 5,770,559 and 5,981,474; Lieberman et al, Lipophilic complexes of pharmacologically active inorganic mineral acid esters of organic compounds, U.S. Pat. No. 5,002,936; Gen, Formative agent of protein complex, US 2002/0119946 A1; Goldenberg et al, Sustained release formulations, WO 2005/105057 A1).

One can dilute the inventive compositions or increase the volume of the pharmaceutical compositions of the invention with an inert material. Such diluents can include carbohydrates, especially, mannitol, α-lactose, anhydrous lactose, cellulose, sucrose, modified dextrans and starch. Certain inorganic salts may also be used as fillers, including calcium triphosphate, magnesium carbonate and sodium chloride. Some commercially available diluents are Fast-Flo, Emdex, STA-Rx 1500, Emcompress and Avicell.

A variety of conventional thickeners are useful in creams, ointments, and gel configurations of the pharmaceutical composition, such as, but not limited to, alginate, xanthan gum, or petrolatum, may also be employed in such configurations of the pharmaceutical composition of the present invention. A permeation or penetration enhancer, such as polyethylene glycol monolaurate, dimethyl sulfoxide, N-vinyl-2-pyrrolidone, N-(2-hydroxyethyl)-pyrrolidone, or 3-hydroxy-N-methyl-2-pyrrolidone can also be employed. Useful techniques for producing hydrogel matrices are known. (E.g., Feijen, Biodegradable hydrogel matrices for the controlled release of pharmacologically active agents, U.S. Pat. No. 4,925,677; Shah et al, Biodegradable pH/thermosensitive hydrogels for sustained delivery of biologically active agents, WO 00/38651 A1). Such biodegradable gel matrices can be formed, for example, by crosslinking a proteinaceous component and a polysaccharide or mucopolysaccharide component, then loading with the inventive composition of matter to be delivered.

Liquid pharmaceutical compositions of the present invention that are sterile solutions or suspensions can be administered to a patient by injection, for example, by subconjunctival injection. The inventive composition can be included in a sterile solid pharmaceutical composition, such as a lyophilized powder, which can be dissolved or suspended at a convenient time before administration to a patient using sterile water, saline, buffered saline or other appropriate sterile injectable medium.

One can dilute or increase the volume of the compound of the invention with an inert material. These diluents could include carbohydrates, especially mannitol, a-lactose, anhydrous lactose, cellulose, sucrose, modified dextrans and starch. Certain inorganic salts can also be used as fillers including calcium triphosphate, magnesium carbonate and sodium chloride. Some commercially available diluents are Fast-Flo™, Emdex™, STA-Rx™ 1500, Emcompress™ and Avicell™.

Disintegrants can be included in the formulation of the pharmaceutical composition into a solid dosage form. Materials used as disintegrants include but are not limited to starch including the commercial disintegrant based on starch, Explotab™. Sodium starch glycolate, Amberlite™, sodium carboxymethylcellulose, ultramylopectin, sodium alginate, gelatin, orange peel, acid carboxymethyl cellulose, natural sponge and bentonite can all be used. Insoluble cationic exchange resin is another form of disintegrant. Powdered gums can be used as disintegrants and as binders and these can include powdered gums such as agar, Karaya or tragacanth. Alginic acid and its sodium salt are also useful as disintegrants.

To aid dissolution of the compound of this invention into the aqueous environment a surfactant might be added as a wetting agent. Surfactants can include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

In one embodiment, the pharmaceutically acceptable carrier can be a liquid and the pharmaceutical composition is prepared in the form of a solution, suspension, emulsion, syrup, elixir or pressurized composition. The active ingredient(s) (e.g., the inventive composition of matter) can be dissolved, diluted or suspended in a pharmaceutically acceptable liquid carrier such as water, an organic solvent, a mixture of both, or pharmaceutically acceptable oils or fats. The liquid carrier can contain other suitable pharmaceutical additives such as detergents and/or solubilizers (e.g., Tween 80, Polysorbate 80), emulsifiers, buffers at appropriate pH (e.g., Tris-HCl, acetate, phosphate), adjuvants, anti-oxidants (e.g., ascorbic acid, sodium metabisulfite), preservatives (e.g., Thimersol, benzyl alcohol), sweeteners, flavoring agents, suspending agents, thickening agents, bulking substances (e.g., lactose, mannitol), colors, viscosity regulators, stabilizers, electrolytes, osmolutes or osmo-regulators. Additives can also be included in the formulation to enhance uptake of the inventive composition. Additives potentially having this property are for instance the fatty acids oleic acid, linoleic acid and linolenic acid.

The composition of this invention can be included in the formulation as fine multiparticulates in the form of granules or pellets of particle size about 1 mm.

Colorants can all be included. For example, the HDACi (or derivative) can be formulated (such as by liposome or microsphere encapsulation).

The powders and tablets preferably contain up to 99% of the active ingredient(s). Suitable solid carriers include, for example, calcium phosphate, magnesium stearate, talc, sugars, lactose, dextrin, starch, gelatin, cellulose, polyvinylpyrrolidine, low melting waxes and ion exchange resins.

Controlled release formulation can be desirable. The composition of this invention can be incorporated into an inert matrix that permits release by either diffusion or leaching mechanisms e.g., gums. Slowly degenerating matrices can also be incorporated into the formulation, e.g., alginates, polysaccharides. Another form of a controlled release of the compositions of this invention is by a method based on the Oros™ therapeutic system (Alza Corp.), i.e., the drug is enclosed in a semipermeable membrane which allows water to enter and push drug out through a single small opening due to osmotic effects. Some enteric coatings also have a delayed release effect.

Pharmaceutically acceptable excipients include carbohydrates such as trehalose, mannitol, xylitol, sucrose, lactose, and sorbitol. Other ingredients for use in formulations can include DPPC, DOPE, DSPC and DOPC. Natural or synthetic surfactants can be used. PEG can be used (even apart from its use in derivatizing the protein or analog). Dextrans, such as cyclodextran, can be used. Cellulose derivatives can be used. Amino acids can be used, such as use in a buffer formulation.

Also, the use of liposomes, microcapsules or microspheres, inclusion complexes, or other types of carriers is contemplated.

Formulations suitable for use with a nebulizer, either jet or ultrasonic, for spraying onto the eye, will typically comprise the inventive compound dissolved in water at a concentration of about 0.1 to 25 mg of biologically active molecule per mL of solution. The formulation can also include a buffer and a simple sugar (e.g., for protein stabilization and regulation of osmotic pressure). The nebulizer formulation can also contain a surfactant, to reduce or prevent surface induced aggregation of the protein caused by atomization of the solution in forming the aerosol.

Dosages. The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

In other embodiments, these methods and pharmaceutical compositions provide therapeutic or prophylactic concentrations of the respective HDACi following administration to a patient. The respective HDACi, e.g., SAHA, may be administered in an amount and using a dosing schedule as appropriate for treatment of a particular disease. In one embodiment, as set forth herein a single dose of the HDACi is provided prior to the surgical event. In addition to a single dose, daily, weekly, bi-weekly, monthly, and bi-monthly doses of the HDACi are contemplated herein; and may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. An appropriate dose and the frequency of dosing may be determined based on several factors, including, for example, the body weight and/or condition of the patient being treated, the severity of the disease being treated, the incidence and/or severity of side effects, the manner of administration, and the judgment of the prescribing physician. Accordingly, appropriate dose ranges may be determined by methods known to those skilled in the art.

Purity of Water and other Ingredients. The water and all other ingredients that are used to make the inventive pharmaceutical composition are preferably of a level of purity meeting the applicable legal or pharmacopoeial standards required for such pharmaceutical compositions and medicaments in the jurisdiction of interest, e.g., United States Pharmacopeia (USP), European Pharmacopeia, Japanese Pharmacopeia, or Chinese Pharmacopeia, etc. For example, according to the USP, Water for Injection is used as an excipient in the production of parenteral and other preparations where product endotoxin content must be controlled, and in other pharmaceutical applications, such as cleaning of certain equipment and parenteral product-contact components; and the minimum quality of source or feed water for the generation of Water for Injection is Drinking Water as defined by the U.S. Environmental Protection Agency (EPA), EU, Japan, or WHO.

Before administration to a patient, the inventive formulations should meet the applicable legal or pharmacopoeial standards required for such pharmaceutical compositions and medicaments in the jurisdiction of interest as to sterility, lack of endotoxin or viral contaminants, etc.

The following working examples are illustrative and not to be construed in any way as limiting the scope of the invention.

### EXAMPLES

### Example 1. HDAC Inhibitor (HDACi) Prevents Excessive Wound Healing and Scar Formation in Rabbit Model of Glaucoma Filtration Surgery

### Material and Methods

### Background

Briefly, a rabbit model of glaucoma filtration surgery (GFS) was used. Rabbits underwent GFS received Balanced Salt Solution (BSS) or SAHA (50 μM) or mitomycin C (MMC; 0.04% (w/v)). Clinical scores of intraocular pressure (TOP), bleb vascularity and slit lamp examination were performed. On postoperative day 14, rabbits were sacrificed and the bleb tissues were collected for evaluation of tissue fibrosis with H&E, Masson trichrome (MT), α-smooth muscle actin (α-SMA), and F-actin staining. Further, SAHA-mediated acetylation of histones in corneal fibroblasts and conjunctiva were determined by western blot analysis.

### Preparation of SAHA Solution and Treatment Regimen.

A 10 mM stock solution of SAHA (Cayman Chemical Company, Ann Arbor, Mich., USA) was prepared by dissolving in dimethyl sulfoxide (DMSO) and then further diluted to 50 μM with balanced salt solution (BSS) eye drops (Alcon). For vehicle control, the same volume of DMSO was diluted with BSS. The 0.04% MMC stock solution was prepared in BSS and 0.02% final dose of MMC was given to rabbits. The rabbits were divided into three treatment groups. Groups I rabbits received 100 μl subconjunctival injection of vehicle 30 minutes before the GFS surgery and group II rabbits received 100 μl subconjunctival injection of 50 μM SAHA solution 30 minutes before GFS. Group III rabbits were injected with 100 μl subconjunctival solution of 0.02% MMC 30 minutes before surgery.

### Clinical Evaluation.

Clinical evaluation was performed to check the intraocular pressure (TOP), general appearance and vascularity of the bleb. All these clinical parameters were recorded before the surgery to obtain the baseline values and on days 3, 5, 7, 10 and 14 after surgery. To measure TOP, tonometry was performed using an applanation tonometer (Tono-pen) with animals under topical anesthesia. Bleb's size was graded by measuring its width and length. Bleb vascularity was graded as 0=avascular; 1=normal vascularity; 2=hyperemic; 3=very hyperemic. Anterior chamber inflammation was assessed by slit lamp examination and graded 0=no inflammation; 1=cells present; 2=fibrin formation; 3=hypopyon present.

### Histological Evaluation.

On postoperative day 14, rabbits were humanely euthanized with pentobarbital (150 mg/kg) under general anesthesia. The eyes were enucleated together with the conjunctiva to preserve the bleb and snap frozen in optimal cutting temperature fluid. The tissues were sectioned and stained with hematoxylin and eosin. Masson's trichrome staining was performed to stain collagen. Immunofluorescence staining for f-actin (a marker for activated fibroblasts and myofibroblasts) was performed using Alexa Fluor® 594-conjugated phalloidin (1:40 dilution, A12381, Invitrogen Inc., Carlsbad, Calif.). Immunofluorescence staining for α-smooth muscle actin (αSMA), a marker for myofibroblasts, was performed with mouse monoclonal primary αSMA antibody (1:100 dilution, M0851; Dako, Carpinteria, Calif.). Tissue sections were incubated with 2% bovine serum albumin for 30 minutes at room temperature and then with αSMA monoclonal antibody for 90 minutes. For detection of the primary antibody, the sections were exposed to Alexa 488 goat anti-mouse IgG secondary antibody (1:500 dilution, A11001; Invitrogen Inc.) for 1 hour. The tissue sections at the site of sclerotomy and the sections on every sixth tissue slide on the either side of the sclerotomy were stained for f-actin and αSMA. After completion of immunostaining, tissue sections were mounted in medium containing DAPI (Vectashield; Vector Laboratories, Inc. Burlingame, Calif.), viewed, and photographed under a fluorescence microscope (Leica, Deerfield, Ill.) equipped with a digital camera system (SpotCam RT KE; Diagnostic Instruments, Sterling, Mich.). The stained areas in the imaged slides were quantified using Image J software.

### Western Blot Analyses.

Human corneal fibroblast cells were treated with or without SAHA (2.5 μM) at different time intervals as indicated in figure legends. Rabbits were received 100 μl subconjunctival injection of 50 μM SAHA. Cell lysates were prepared from human corneal fibroblast cells treated with SAHA (2.5 μM) at 0, 2, 4, 6, 16 and 24 hours. Rabbit conjunctival tissues were harvested at 0, 2, 6 and 24 hour time points. Cell and tissue lysates were analyzed by western blotting using anti-acetyl Histone H3, anti-acetyl histone H4 (Cell Signaling, Beverly, Mass., USA) and (3-actin (Santa Cruz biotechnology Inc., Dallas, Tex., USA) antibodies respectively. All western blots for each protein were used 3 rabbit tissues and repeated at least two times. Digital quantification of western blots was performed using NIH Image J software and Image Studio software Version 5.2

### Statistical Analysis.

The results are expressed as mean±SEM. The data for bleb length, area, vascularity and IOP were analyzed by two way ANOVA and Bonferroni test using GraphPad Prism 6.0 (GraphPad Software, Inc., La Jolla, Calif.) and p<0.05 was considered to be statistical significant. The immunostaining data was analyzed using a t-test.

### Results

### Clinical Evaluation.

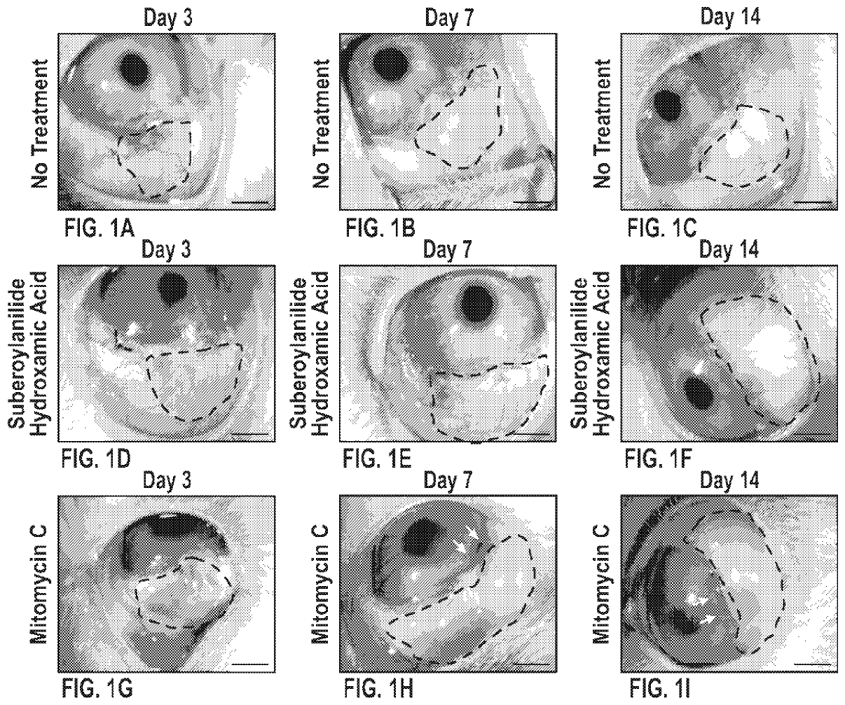

The subconjunctival injection of SAHA was well tolerated, and no signs of hyperemia or inflammation were detected at the site of injection. No sign of corneal edema, corneal opacity, endophthalmitis, or cataract were observed in any of the SAHA-treated rabbits. On the contrary, MMC-treated rabbit eyes showed corneal neovascularization and opacity (FIG. 1H and FIG. 1I).

### Bleb Morphology and Characteristics.

Biomicroscopy was performed on the rabbit eyes to monitor bleb characteristics. FIG. 1 shows the typical appearance of blebs in no treatment control (FIG. 1A-C), SAHA (FIG. 1D-F) and MMC-treated (FIG. 1G-I) rabbits. SAHA-treated rabbit eyes showed transparent and elevated blebs (FIG. 1D-F) compared to flat and scarred blebs in no treatment control rabbits (FIG. 1A-C). The MMC-treated rabbits also showed elevated blebs but these blebs had a thin, avascular and cystic appearance (FIG. 1G-I).

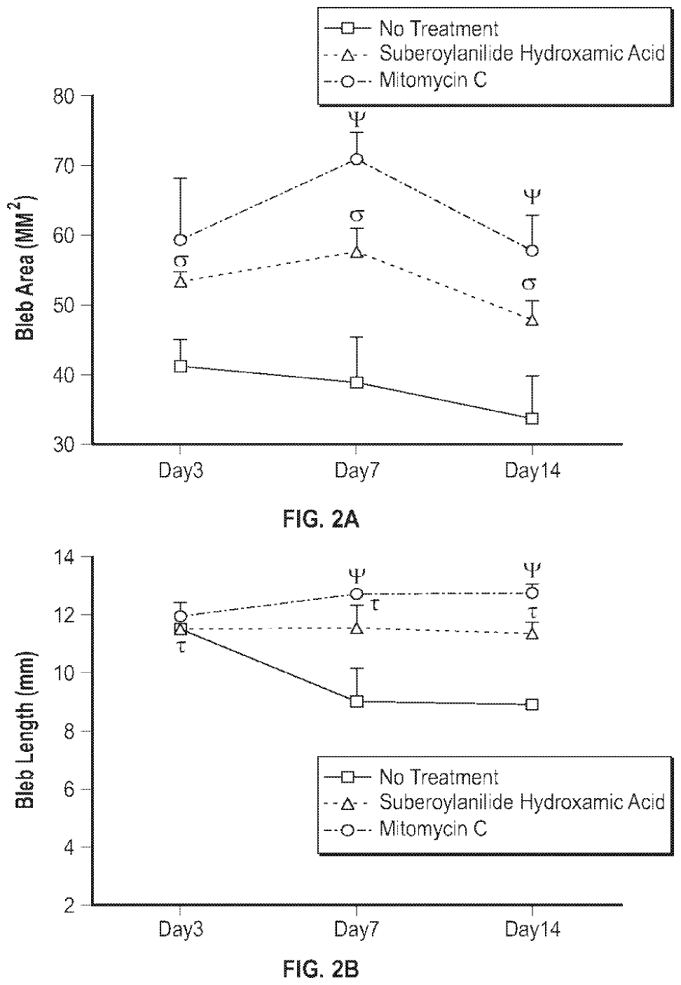

The morphometric analysis of the bleb length and area was performed using Vernier caliper and digital quantification of the acquired images. FIG. 2 shows the mean bleb length (FIG. 2A) and bleb area (FIG. 2B) in no treatment, SAHA-treated and MMC-treated rabbits at day 3, day 7 and day 14 after GFS. The SAHA-treated rabbit eyes showed significantly higher bleb area (p<0.05) and length (p<0.001) compared to untreated controls (FIG. 2A-B). The MMC-treated rabbit eyes also showed significantly higher bleb area (p<0.01) and length (p<0.001) as compared to untreated controls. The relative comparison between SAHA- and MMC-treated groups demonstrated that MMC-treated rabbits had higher bleb area and length as compared to SAHA-treated rabbits but the difference was not statistically significant.

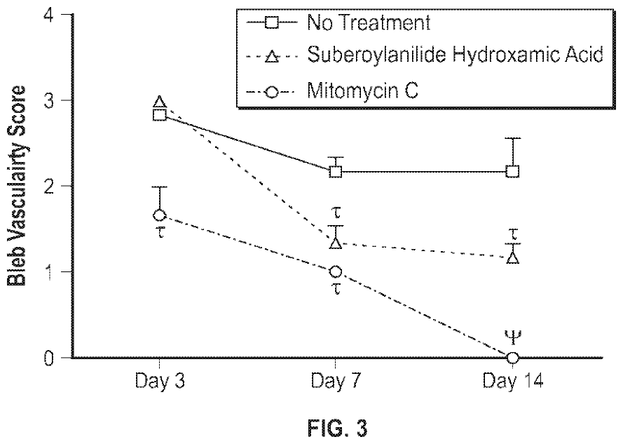

FIG. 3 shows vascularity scores of the blebs in no treatment control, SAHA-treated and MMC-treated rabbits. The no treatment control group showed increased vascularity in response to the surgical trauma. SAHA-treatment significantly (p<0.01) attenuated bleb vascularity on day 7 and day 14 after glaucoma filtration surgery. MMC-treatment also caused a very robust decrease in bleb vascularity and the effect was significantly more as compared to SAHA. By day 14, all the MMC-treated blebs were completely avascular and had a cystic appearance.

### Intraocular Pressure.

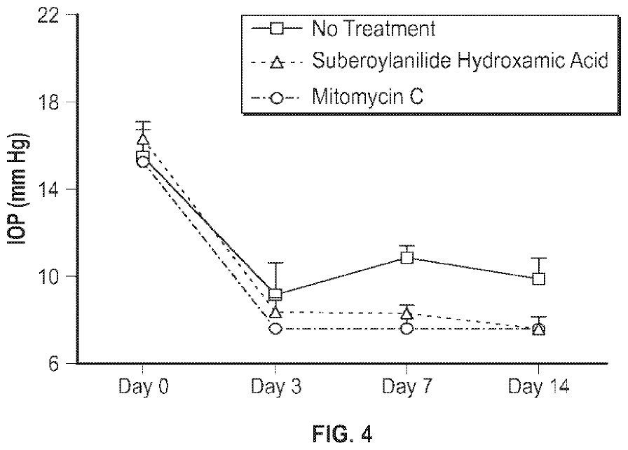

FIG. 4 shows the effect of SAHA- and MMC-treatment on intraocular pressure (TOP) in the rabbit eyes after GFS. As anticipated, there was a significant (p<0.01) decrease in TOP in the no-treatment control, SAHA- and MMC-treated rabbits on day 3 after GFS as compared to the preoperative baseline values, indicating successful surgery. However, by day 7 and day 14 the TOP started to increase in the untreated control group. On the contrary, SAHA- and MMC-treated rabbit showed lower TOP compared to the no-treatment control eyes but the results were not statistically significant.

### Histological Evaluation.

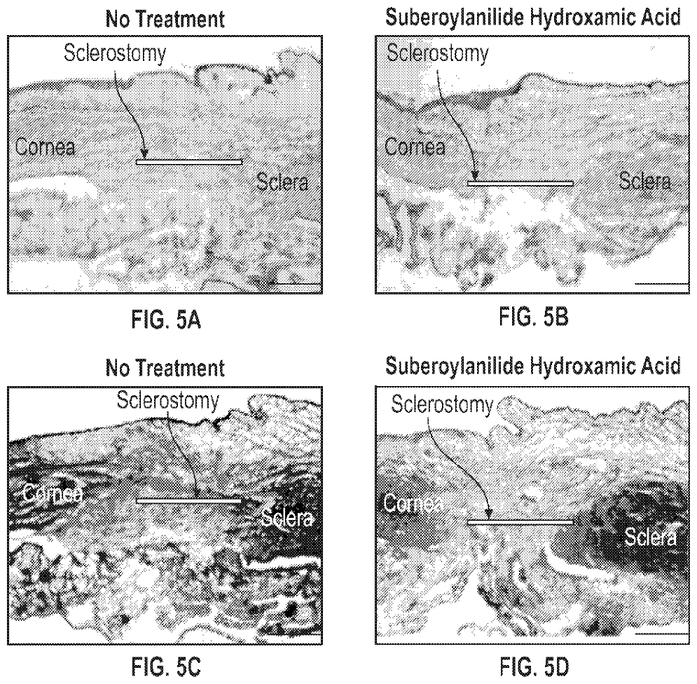

To evaluate whether SAHA-treatment affected collagen deposition and myofibroblast formation after GFS, histological and immunostaining was performed on the rabbit eye tissue sections. The haemotoxylin and eosin (H&E) staining of the eye tissues obtained from no-treatment control rabbits (FIG. 5A) shows that the site of sclerotomy was densely packed with fibrous tissue. On the contrary, eye tissues of SAHA-treated rabbits (FIG. 5B) showed mild fibrous deposit and a loosely arranged conjunctival tissue. Further, Masson trichrome staining revealed less collagen deposits at the site of sclerotomy in the SAHA-treated (FIG. 5D) tissues as compared to the eye tissue sections obtained from no-treatment control rabbits (FIG. 5C).

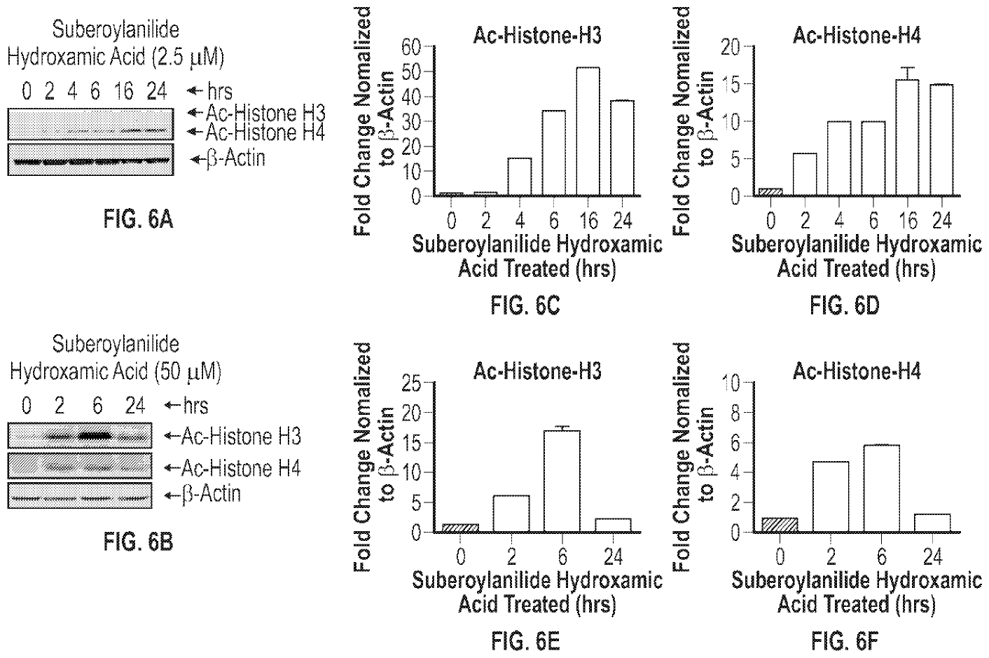

To determine the effect of SAHA-treatment in acetylation status of histones, we performed western blot analyses using human corneal fibroblast cells (FIG. 6A) and conjunctiva tissue (FIG. 6B) treated with SAHA at different time intervals. SAHA-treatment increase the acetylation status of histone H3 and H4 and attained maximum at 6 hours and gradual decrease in 24 hrs. Also, corresponding western blot quantitation data is provided in FIG. 6C-F. These data suggest that SAHA-treatment effectively increase the acetylation status of Histone H3 and H4 thereby regulating target gene expression or repression involved in excessive wound healing and scar formation.

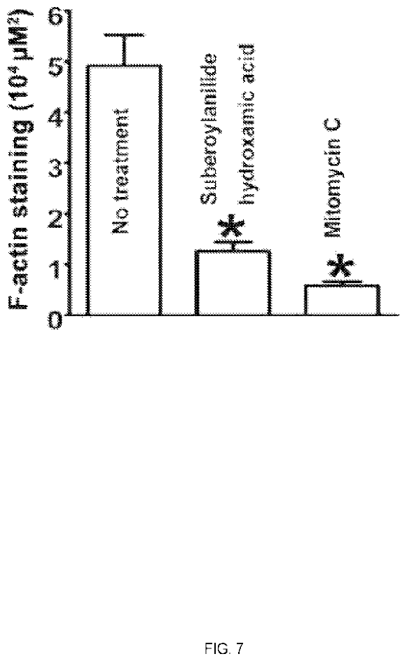

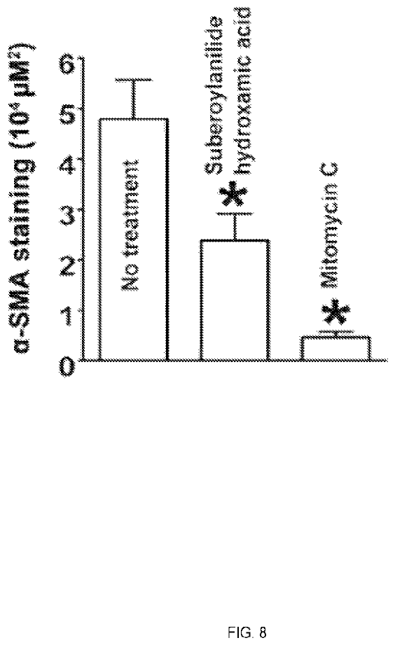

Presence of activated fibroblasts and myofibroblasts is a key feature of scarred bleb. Therefore, immunostaining for f-actin (a marker for activated fibroblasts and myofibroblasts) and αSMA (a marker for myofibroblasts) was performed to detect the changes in the pattern of these proteins. Ocular tissue sections collected from no-treatment control rabbits showed intense f-actin and αSMA staining at the site of sclerostomy, in the subconjunctival space and in the sclera. On the other hand, ocular tissues collected from SAHA-treated rabbit showed sparse f-actin and αSMA staining at the site of sclerostomy, in the subconjunctival space and in the sclera. Morphometric quantification for f-actin and αSMA revealed that SAHA-treatment caused a significant decrease in the f-actin (FIG. 7) and αSMA (FIG. 8) stained area (p<0.01), thus confirming that the improved bleb characteristics in SAHA-treated rabbits is complemented by a decreased fibrosis and scarring at the site of sclerostomy.

As expected, MMC-treatment also caused a very robust decrease in f-actin and αSMA staining. The morphometric quantification revealed that the MMC effect was significantly more as compared to SAHA. However, it should be noted that MMC-treatment was associated with notable toxicity to the conjunctival epithelium. The morphometric quantification of DAPI nuclear staining revealed (FIG. 7 and FIG. 8) a continuous and uniform conjunctival epithelium at the site of sclerostomy in no-treatment control and SAHA-treated rabbit tissue sections. On the other hand, MMC-treated rabbit tissue showed discontinuous and highly sparse DAPI nuclear staining for the conjunctival epithelium, thus indicating a cytotoxic effect of MMC to the cells of conjunctival epithelium.

In summary, SAHA-treatment after glaucoma filtration surgery showed no signs of edema, corneal opacity, endophthalmitis or cataract formation. Morphometric analysis of SAHA-treated eyes showed higher bleb length (p<0.001), bleb area (p<0.05), lower TOP (p<0.01) and decreased vascularity compared to control. Further, SAHA-treatment showed significantly reduced levels of αSMA (p<0.001), F-actin (p<0.01), and collagen deposition (p<0.05) at the sclerotomy site. In addition, SAHA-treatment increased the acetylation status of H3 and H4 histones in corneal fibroblasts and conjunctiva.

### Example 2. HDAC Inhibitor (HDACi) Prevents Corneal Haze Formation Long-Term, after Photorefractive Keratectomy (PRK) Surgery

### Material and Methods

### Background.

Briefly, corneal haze in rabbits was produced with −9.0 diopter PRK. A single application of SAHA (25 μM) or MMC (0.02%) was applied topically immediately after PRK. Effects of the two drugs were analyzed by slit-lamp microscope, specular microscope, TUNEL assay, and immunofluorescence.

### Corneal Haze Production in Rabbit Eyes.

Photorefractive keratectomy was used to produce corneal haze in rabbits by performing −9.0 diopter ablation with the Summit Apex excimer laser (Model: SVS APEX Plus ER; Alcon, Ft. Worth, Tex.) as reported previously. Briefly, the rabbits were anesthetized and local anesthesia of the cornea was achieved through the application of topical ophthalmic 0.5% proparacaine hydrochloride (Alcon, Fort Worth, Tex.). A wire lid speculum was placed and corneal epithelium was removed by gentle scraping with surgical Beaver blade #64 (BD Biosciences, Franklin Lakes, N.J.). Spherical laser ablation of −9.0 diopters, with a 6.0 mm-diameter optical zone, was performed by programming the laser (421 pulses for a depth of 108 μm). This treatment is known to produce significant haze in rabbit cornea with 100% reproducibility. This technique consistently produces corneal haze and myofibroblasts in the rabbit cornea which peaks at 4 weeks post-PRK. Only one eye of each animal was used for experimentation.

### SAHA and MMC Treatment Regimen.

A 10 mM stock solution of SAHA (Cayman Chemical Company, Ann Arbor, Mich., USA) was prepared using dimethyl sulfoxide (DMSO), and diluted to 25 μM with balanced salt solution (BSS) eye drops (Alcon). For vehicle control, the same volume of DMSO was diluted with BSS. The 0.02% Mitomycin C solution was prepared at the Harry S. Truman VA Hospital Pharmacy, Columbia, Mo. using 5 mg/ml powder (Accord Healthcare, Inc., Durham, N.C., USA) diluted with sterile normal saline. This solution was stable for 1 week at room temperature but was used within 48 hours after preparation. After PRK, rabbits were divided into 3 groups: Group-1 received a single topical application of SAHA (25 μM) for 5 minutes (n=15), Group-2 received a single topical application of MMC (0.02%) for 1 minute (n=15) and Group-3 received a single topical application of vehicle for 5 minutes (n=15). Thereafter, eyes were washed profusely with BSS. The contralateral eye served as naive control. All rabbits received clinical eye examination.

### Slit-lamp and Specular Biomicroscopy.

Slit-lamp (SL15, Kowa, Japan), stereo (MZ16F, Leica, Switzerland) and specular (NSP-8800, Konan, Japan) microscopes were used to evaluate ocular health, corneal haze, and corneal endothelial cells in anesthetized animals. Grading of clinical corneal haze was done using Fantes scale by three researchers (AS, MW and SG) in a masked manner. High performance digital imaging system (VK2; Kowa Tokyo, Japan) was used for corneal image analysis.

### Cornea Collection, Immunofluorescence and TUNEL Assay.

Rabbits from each group were euthanatized 3-days (n=3), 1-month (n=6), and 4-months (n=6) after PRK. Corneas were snap frozen, sectioned (7 mm), and stored at −80° C. Immunofluorescence was used to detect myofibroblast marker αSMA using monoclonal antibody (M0851; Dako, Calif.). TUNEL assay (ApopTag, 57165; Billerica, Mass.) detected apoptosis and DAPI-stained nuclei determined cellular density. Sections were viewed and photographed under a fluorescence microscope (Leica, Deerfield, Ill.) equipped with a digital camera system (SpotCam RT KE; Diagnostic Instruments, MI).

### Quantification and Statistical Analyses.

Stained proteins in corneal sections were quantified using Image J software in six randomly selected areas. Standard error means were calculated. One-way ANOVA and Wilcoxon rank sum test were used for statistical analysis. The p value <0.05 was considered significant.

### Results

### In Vivo Efficacy Studies of SAHA and MMC.

Biomicroscopy images showed levels of corneal haze in live rabbits. A single application of SAHA or MMC significantly decreased post-PRK corneal haze in rabbits compared to no-drug treated corneas (p<0.001). Haze score at 4-months in BSS-treated rabbit corneas was elevated (2.7±0.4) compared to SAHA-treated corneas (0.9±0.1), and MMC-treated corneas (0.85±0.15).

The inhibitory effects of SAHA and MMC in the development of myofibroblasts post-PRK in rabbit corneas were determined at 1-month and 4-months measured with αSMA immunofluorescence. Rabbit corneas that received a single topical application of SAHA or MMC after PRK showed significantly less α-SMA+ cells (70-93%; P<0.001) in the stroma at 1-month and 4-months compared to the BSS controls.

### In Vivo Safety Analyses of SAHA and MMC.

The effects of SAHA and MMC application on rabbit corneal endothelial cell phenotype and density in vivo at 1-month and 4-months after −9D PRK were determined. As an indicator of safety, the images showed that SAHA treatment does not alter endothelial cell phenotype or density. At 1-month after PRK-performed, BSS or SAHA treated rabbit eyes exhibited classical endothelial polygonal mosaic phenotype with few occasional large normal hexagonal endothelial cells, whereas MMC-treated corneas exhibited significantly compromised endothelial cell phenotype and density. Similar pattern was noted at 4-months where BSS or SAHA treated corneas showed normal endothelium and MMC-treated corneas still had compromised endothelial cells.

The effects of SAHA and MMC application on corneal keratocyte number and density in vivo at 1-month and 4-months after −9D PRK analyzed with DAPI-staining was determined. Corneas treated with MMC demonstrated significant keratocyte loss within the anterior stroma at 1-month and 4-months post-PRK. Conversely, SAHA did not reduce number or density of keratocytes in rabbit stroma at these time points and showed cellular density similar to BSS-treated corneas.

The effects of SAHA and MMC on corneal keratocyte apoptosis measured by TUNEL assay at 3-days, 1-month and 4-months was determined. Treatment with BSS or SAHA did not induce apoptosis in any cell type, while treatment with MMC caused significant apoptosis in epithelial and keratocyte cells of the rabbit corneas collected at 3-days. Similarly, the long-term toxicity studies of 1-month and 4-months detected many TUNEL+ cells in the stroma and endothelium of corneas treated with MMC but not in corneas treated with SAHA.

What is claimed is:

1. A method of promoting wound healing with reduced scarring after glaucoma filtration surgery in a mammalian subject in need thereof, comprising administering an effective amount of a HDAC inhibitor (HDACi) to said subject.

2. The method of claim 1, wherein the HDACi is suberoylanilide hydroxamic acid (SAHA), or a derivative thereof.

3. The method of claim 2, wherein fibroblast migration and activation is inhibited.

4. The method of claim 2, wherein myofibroblast formation is inhibited.

5. The method of claim 4, wherein myofibroblast formation is inhibited while preserving cell viability.

6. The method of claim 1, wherein the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

7. A pharmaceutical composition, comprising a HDACi, and a pharmaceutically acceptable carrier or excipient suitable for ophthalmic use.

8. The pharmaceutical composition of claim 7, wherein the HDACi is suberoylanilide hydroxamic acid (SAHA) or a derivative thereof, or a physiologically acceptable salt thereof.

9. The pharmaceutical composition of claim 7, wherein the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.

10. A method of preventing or reducing corneal haze formation long-term, after photorefractive keratectomy (PRK) surgery in a mammalian subject in need thereof, comprising administering an effective amount of a HDAC inhibitor (HDACi) to said subject, wherein said corneal haze formation is prevented or reduced long-term.

11. The method of claim 10, wherein the HDACi is suberoylanilide hydroxamic acid (SAHA), or a derivative thereof.

12. The method of claim 11, wherein said corneal haze formation is prevented or reduced for a period selected from the group consisting of: greater than 1 month; greater than or equal to 2 months, greater than or equal to 3 months, greater than or equal to 4 months, greater than or equal to 5 months, greater than or equal to 6 months, greater than or equal to 7 months, greater than or equal to 8 months, greater than or equal to 9 months, greater than or equal to 10 months, greater than or equal to 11 months, greater than or equal to 12 months.

13. The method of claim 12, wherein said corneal haze formation is prevented or reduced for a period greater than or equal to 4 months, and the endothelial cell phenotype and density is not compromised.

14. The method of claim 12, wherein said corneal haze formation is prevented or reduced for a period greater than or equal to 4 months, and the density of keratocytes is not reduced.

15. The method of claim 10, wherein the HDACi is selected from the group consisting of Entinostat (MS-275); Panobinostat (LBH589); Trichostatin A (TSA); Mocetinostat (MGCD0103); Belinostat (PXD101); Romidepsin (FK228, Depsipeptide); MC1568; Tubastatin A HCl; Givinostat (ITF2357); Dacinostat (LAQ824); CUDC-101; Quisinostat (JNJ-26481585); Pracinostat (SB939); PCI-34051; Droxinostat; Abexinostat (PCI-24781); RGFP966; AR-42; Ricolinostat (ACY-1215); Tacedinaline (CI994); CUDC-907; M344; Tubacin; RG2833 (RGFP109); Resminostat; Tubastatin A; WT161; ACY-738; Tucidinostat (Chidamide); TMP195; (ACY-241); BRD73954; BG45; 4SC-202; CAY10603; LMK-235; CHR-3996; Splitomicin; Santacruzamate A (CAY10683); Nexturastat A; TMP269; HPOB; Valproic acid sodium salt (Sodium valproate), and derivatives of any of these members, or a physiologically acceptable salt of any of these members.
